(19) United States
(12) Patent Application Publication
(10) Pub. No.: US 2024/0352366 A1
(43) Pub. Date: Oct. 24, 2024

(54) PLANT AND PROCESS FOR PRODUCTION OF PURIFIED METHANE

(71) Applicant: L'Air Liquide, Societe Anonyme pour l'Etude et l’Exploitation des Procedes Georges Claude, Paris (FR)

(72) Inventors: Romain LOEB, Sassenage (FR); Jean-Marc BERNHARDT, Sassenage (FR); Golo ZICK, Sassenage (FR)

(73) Assignee: L'Air Liquide, Societe Anonyme pour l'Etude et l’Exploitation des Procedes Georges Claude, Paris (FR)

(21) Appl. No.: 18/639,366

(22) Filed: Apr. 18, 2024

(30) Foreign Application Priority Data
Apr. 20, 2023 (FR) FR 2303953

(51) Int. Cl. C10L 3/08; B01D 53/22; C07C 7/00; C07C 7/144; C10L 3/10

(52) U.S. Cl. CPC C10L 3/08; B01D 53/22; C07C 7/005; C07C 7/144; C10L 3/104; B01D 2256/245; B01D 2257/504; B01D 2258/05; C10L 2290/548

(57) ABSTRACT

A plant for production of purified methane from natural gas and biogas, having, arranged in series in a gas circuit, a heavy hydrocarbon removal unit and a CO2 removal unit. The plant includes a biogas purification unit having an inlet intended to be connected to a biogas source and a first purified biogas outlet connected to the first inlet of the CO2 removal unit, the biogas purification unit being configured to produce biogas or biomethane with a variable/defined CO2 concentration, and
a boiler having a first inlet connected to the second outlet of the heavy hydrocarbon removal unit, the boiler being configured to produce a thermal power determined as a function of the amount of hydrocarbons removed and supplied by the heavy hydrocarbon removal unit, the boiler being fluidically connected to the CO2 removal unit to provide said unit with thermal energy produced for CO2 removal.

### CROSS-REFERENCE TO RELATED APPLICATIONS

This application claims the benefit of priority under 35 U.S.C. § 119 (a) and (b) to French patent application No. FR 2 303 953, filed Apr. 20, 2023, the entire contents of which are incorporated herein by reference.

### BACKGROUND

The present invention relates to a plant and a process for production of purified methane.

### FIELD OF THE INVENTION

The invention relates more particularly to a plant for production of purified methane from natural gas and biogas, comprising, arranged in series in a gas circuit, a heavy hydrocarbon removal unit and a CO2 removal unit.

### RELATED ART

Biogas is the gas produced during the degradation of organic matter in the absence of oxygen (anaerobic fermentation), also known as methanization. This may be a natural degradation that is observed in marshes or household waste landfills. Biogas production may also result from the methanization of waste in a dedicated reactor, the conditions of which are controlled. This reactor is called a methanizer or digester, then in a post-digester, similar to the digester and allowing the methanization reaction to be further advanced.

Biomass is any grouping of organic matter that can be transformed into energy through this methanization process, for example sewage sludge, manure/slurry, agricultural residues, and food waste.

Biogas contains mainly methane (CH4) and carbon dioxide (CO2) in varying proportions depending on the mode of acquisition and the substrate, but may also contain, to a lesser extent, water, nitrogen, hydrogen sulfide (H2S), oxygen, and other organic compounds, in trace amounts, including H2S, between 10 and 50 000 ppmv.

Depending on the organic matter degraded and the techniques used, the proportions of the components differ, but on average biogas comprises (by mole or by volume), on a dry gas basis, 30 to 75% methane, 15 to 60% carbon dioxide, 0 to 15% nitrogen, 0 to 5% oxygen, and compounds in trace form.

Biogas can be enhanced in value in various ways. It can, after a gentle treatment, be enhanced in value near to the production site to provide heat, electricity or a mixture of the two (cogeneration); the high carbon dioxide content reduces its calorific value, increases the costs of compression and of transportation, and limits the economic advantage of treating it in view of this nearby use.

More advanced purification of the biogas enables its wider use; in particular, advanced purification of the biogas makes it possible to obtain a biogas which is purified to the specifications of natural gas and which can be substituted for it. The biogas purified in this way is called “biomethane”. Biomethane thus supplements natural gas resources with a renewable part produced within territories; it can be used for exactly the same uses as natural gas of fossil origin. It can supply a natural gas network or a filling station for vehicles; it can also be liquefied to be stored as liquid natural gas (bio-LNG).

Liquefaction plants for producing bio-LNG from natural gas from the network may in some cases impose a new configuration comprising two streams with different compositions, flow rates and pressures (natural gas and biogas).

Natural gas is usually distributed in high-pressure networks (>40 barg) and has molecules unsuitable for liquefaction. In general, about 1 to 2 mol % CO2 and long carbon chains (heavy hydrocarbons) are observed. Raw biogas is produced at low pressure (>˜ 0 barg) and with a standard composition of 30 to 50 mol % CO2. The rest is mainly composed of methane and some impurities (H2S, etc.). The raw biogas generated is generally improved in biomethane to a CO2 content of 2 to 4 mol % in a process called “upgrading” (separation by membranes or amine scrubbing) and then injected into a network, liquefied or compressed in cylinders for transport (CBG).

To produce methane at the necessary quality required for liquefaction, amine scrubbing solutions or PTSA are commonly used to purify the methane stream comprising 1 to 4 mol % CO2 at the inlet of these units to 50 to 400 ppm, called “polishing”.

Heavy hydrocarbons must be evacuated to avoid them freezing and plugging the main heat exchanger of a liquefaction unit in the same way as CO2 does and thus stopping the liquefaction process. The solutions for removing heavy hydrocarbons are not yet well defined in the market for small liquefaction units, whereas for very large liquefaction units (>100 tpd of natural gas) the expensive solution of cryogenic distillation may be considered.

To produce purified methane of quality having between 0 and 400 ppm, in particular between 50 and 400 ppm, of CO2 from natural gas and biogas sources, the traditional solution is to mix the two incoming streams. The mixing is done after the removal of heavy hydrocarbons for the natural gas stream and after a standard upgrading (purification of raw biogas to produce biomethane containing 2 to 4% CO2) of the biogas with membranes. The mixing is carried out at the standard liquefaction pressure of the biogas, being the pressure achievable in a single compression unit and necessary for membrane purification (about 12 barg). However, this approach has the following drawbacks:
The natural gas must be expanded from the pressure of the network to the pressure of biomethane. A consequent loss of energy (usable energy) would therefore be observed on this stream.
The upgrading operation produces a stream containing a fixed CO2 content (the standard being 2 to 4 mol % at the outlet).
The additional purification operation, or polishing, requires an input of heat for the additional removal of CO2, heat which is supplied by external energy consumption, for example by the burning of natural gas (from the network or taken from the flow entering the plant), by a source of electrical energy, or by a mixture of several energy sources. The additional purification operation can, for example, be scrubbing with amines, to purify the methane to the necessary quality required for liquefaction or polishing, for example having a CO2 content of 50 to 400 ppm.
When upgrading is followed by an additional purification operation, this operation would be carried out on a stream containing a fixed CO2 content, which would not be optimized, i.e. neither adapted nor adaptable, in relation to the amount of energy actually available in situ for the additional purification operation.
The upgrading of raw biogas (purification of raw biogas) has a strong economic impact on the plant since membranes have a high investment cost while the secondary polishing (the purification of the mixture of purified biogas and natural gas to achieve an even lower CO2 level), for example amine scrubbing, requires a high operating cost linked to the heat requirements (proportional to the CO2 level to be removed).
An object of the present invention is to overcome all or part of the drawbacks noted above.

### SUMMARY OF THE INVENTION

The present invention proposes an innovative plant and process for production of purified methane from two different sources of natural gas and biogas that allows the optimization of the methane purification operation. This optimization makes it possible to use the pressure of the natural gas network advantageously to increase the specific efficiency of the liquefier. It is also possible to optimize the biogas purification unit by using the energy available from the heavy hydrocarbons extracted from natural gas at the heavy hydrocarbon removal unit so that the energy required for further CO2 removal is equal to the energy obtained by burning the heavy hydrocarbons.

The plant according to the invention, which also complies with the generic definition thereof given in the preamble above, is essentially characterized in that the heavy hydrocarbon removal unit comprises a first inlet intended to be connected to a gas source comprising natural gas, a first outlet for gas purged of heavy hydrocarbons, and a second outlet for the removed heavy hydrocarbons. The CO2 removal unit comprises a first inlet connected to the first outlet of the heavy hydrocarbon removal unit, a first outlet for gas of reduced CO2 content, and a second outlet for CO2-enriched effluent gases. The plant further comprises:
a biogas purification unit comprising an inlet intended to be connected to a biogas source and a first purified biogas outlet connected to the first inlet of the CO2 removal unit, the biogas purification unit being configured to produce biogas or biomethane with a variable/defined CO2 concentration, and
a boiler for producing heat by combustion of heavy hydrocarbons, comprising a first inlet connected to the second outlet of the heavy hydrocarbon removal unit, the boiler being configured to produce a thermal power determined as a function of the amount of hydrocarbons removed and supplied by the heavy hydrocarbon removal unit, the boiler being fluidically connected to the CO2 removal unit to provide said unit with thermal energy produced for CO2 removal.

In addition, embodiments of the invention may comprise one or more of the following features:
the CO2 removal unit comprises or is an amine scrubbing unit,
the CO2 removal unit is configured to have the capacity to remove a defined amount of CO2 from a gas stream, in particular an amine scrubbing capacity, said capacity being a function of the thermal energy provided by the boiler,
the plant is configured to measure or determine the thermal energy produced by the boiler and provided to the CO2 removal unit,
the plant is configured to modulate the level of purification of the biogas as a function of the thermal energy produced by the boiler and provided to the CO2 removal unit,
the boiler is configured to provide the heavy hydrocarbon removal unit with thermal energy,
the CO2 removal unit comprises a third gas outlet comprising a fuel and the boiler comprises a second fuel gas inlet connected to the third outlet of the CO2 removal unit,
the biogas purification unit is a membrane separation unit,
the plant comprises a conduit intended to connect the first purified biogas outlet to a natural gas network, and
the biogas purification unit comprises a second CO2-rich gas outlet.

The invention also relates to a process for production of purified methane from natural gas and biogas, the process comprising:
a step of supplying gas comprising natural gas to a heavy hydrocarbon removal unit,
a step of removing heavy hydrocarbons from said gas by the heavy hydrocarbon removal unit to produce a gas purged of heavy hydrocarbons,
a step of providing the gas purged of heavy hydrocarbons to a CO2 removal unit, the CO2 removal unit ensuring CO2 removal by a process comprising heating,
a step of supplying a biogas purification unit with biogas,
a step of purifying biogas in the biogas purification unit to produce a purified biogas,
a step of providing the purified biogas to the CO2 removal unit,
a step of burning, in a boiler, the heavy hydrocarbons removed by the heavy hydrocarbon removal unit,
a step of providing the thermal energy produced by the burning of the heavy hydrocarbons to the CO2 removal unit,
a step of removing CO2 within the gas stream consisting of or comprising the sum of the gas purged of heavy hydrocarbons and the purified biogas, the CO2 removal step being configured to reduce the CO2 concentration in the gas stream to a defined threshold,
   in which the biogas purification step is configured to produce a purified biogas having a defined CO2 concentration, said concentration being chosen as a function of the total amount of CO2 to be removed from the gas stream in the CO2 removal unit in order to reach the CO2 concentration threshold.

According to other possible distinguishing features:
the total amount of CO2 to be removed from the gas stream in the CO2 removal unit to reach the CO2 concentration threshold corresponds to the CO2 removal capacity of the removal unit, said capacity being determined by the thermal energy produced and provided by the boiler,
the process comprises a step of determining the thermal energy produced by the boiler and available to the CO2 removal unit, and
the purified biogas is compressed to the pressure of the gas purged of heavy hydrocarbons and combined with said gas to form the gas stream produced to the CO2 removal unit.

The invention may also relate to any alternative device or process comprising any combination of the features above or below within the scope of the claims.

Other specifics and advantages will become apparent from reading the following description, made with reference to the FIGURE.

### BRIEF DESCRIPTION OF THE FIGURES

FIG. 1 represents a schematic and partial view illustrating an embodiment example of the structure and operation of a plant according to the invention.

### DETAILED DESCRIPTION OF THE INVENTION

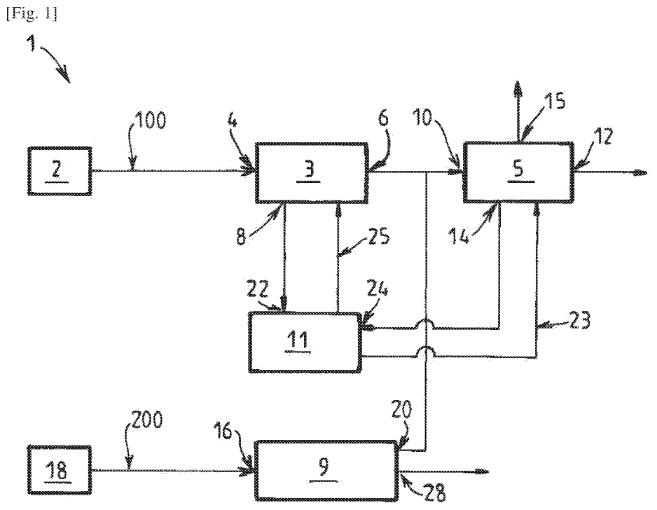

The example of a purified methane production plant illustrated in FIG. 1 comprises, arranged in series in a natural gas circuit 100, a heavy hydrocarbon removal unit 3 and a CO2 removal unit 5.

In one embodiment, the heavy hydrocarbon removal unit 3 comprises, for example, two (or more) adsorbent cylinders arranged in parallel and operating alternately. The adsorbent comprises, for example, carbon filters that can be regenerated by heat. The adsorbent gradually adsorbs heavier carbon species at its adsorption sites. The gas which has been purified, i.e. freed of the compounds which occupy the adsorbent adsorption sites, circulates in a closed loop to regenerate the adsorbent without losses of methane. During the regeneration of the adsorbent of one cylinder, another cylinder goes into heavy hydrocarbon removal operation. The adsorbent is calibrated to trap at least part of the C3 hydrocarbons (propane) and all the heavier hydrocarbons (e.g. butane, pentane, hexane, aromatics (benzene, ethylbenzene, xylene, toluene, etc.), among others). The heavy hydrocarbon removal unit 3 is, for example, a pressure-temperature swing adsorption (PTSA) unit with 2 to 3 adsorbent cylinders arranged in parallel.

In one embodiment, the heavy hydrocarbon removal unit 3 may comprise at least one distillation column comprising a solution for cold distillation of heavy species. The distillation column withdraws a hydrocarbon stream at a temperature of between −10 and −120° C., in particular between −30 and −80° C.

The hydrocarbons removed may be in gaseous or liquid form.

The heavy hydrocarbon removal unit 3 comprises a first inlet 4 intended to be connected to a gas source 2 comprising natural gas (for example gas circulating in a local, regional or national natural gas distribution network). The heavy hydrocarbon removal unit 3 further comprises a first outlet 6 for gas purged of heavy hydrocarbons, and a second outlet 8 for the removed heavy hydrocarbons. The gas source 2 is, for example, a pressurized natural gas network.

The CO2 removal unit 5 comprises a first inlet 10 connected to the first outlet 6 of the heavy hydrocarbon removal unit 3, a first outlet 12 for gas of reduced CO2 content, and a second outlet 15 for CO2-enriched effluent gases.

The plant 1 comprises a biogas purification unit 9 which is arranged in series with the CO2 removal unit 5 in a biogas circuit 200.

The biogas purification unit 9 comprises an inlet 16 intended to be connected to a source 18 of raw biogas, for example a digester, and a first outlet 20 of purified biogas, i.e. biogas with a higher CH4 concentration and a lower CO2 concentration than the raw biogas admitted at the inlet 16. The purified biogas leaving the first outlet 20 has a variable degree of CO2, for example between 2% and 20%.

The first outlet 20 is connected to the first inlet 10 of the CO2 removal unit 5. The biogas purification unit 9 is configured to produce purified biogas or biomethane with a variable/defined CO2 concentration.

The biogas purification unit 9 may comprise any device capable of producing methane-enriched gas from raw biogas, the power of which can be regulated to produce a methane-enriched gas with a variable/defined CO2 concentration. The biogas purification unit 9 can operate according to the principle of separation and/or adsorption and/or absorption. It may for example comprise a pressure swing adsorption (PSA), amine scrubbing, water scrubbing, organic physical scrubbing, cryogenic distillation or membrane separation system.

Preferably, the biogas purification unit 9 is a membrane permeation treatment unit (or a membrane separation unit).

The plant 1 comprises a boiler 11 for producing heat by burning heavy hydrocarbons. The boiler 11 comprises a first inlet 22 connected to the second outlet 8 of the heavy hydrocarbon removal unit 3. The first inlet 22 of the boiler 11 is configured to supply the boiler 11 with heavy hydrocarbons which exit via the second outlet 8 of the heavy hydrocarbon removal unit 3. The boiler 11 is configured to produce a thermal power determined as a function of the quantity of hydrocarbons removed and provided by the heavy hydrocarbon removal unit 3. The boiler 11 is connected 23, for example by means of a heat transfer fluid, to the CO2 removal unit 5 to provide the latter with thermal energy produced for the removal of CO2.

The CO2 removal unit 5 is configured to have the capacity to remove a defined amount of CO2 from a gas stream which is a function of the thermal energy provided by the boiler 11.

The thermal energy produced by the burning of heavy hydrocarbons in the boiler 11 provides the thermal energy necessary for the operation of the CO2 removal unit 5. That is, the CO2 removal capacity or the amount of CO2 that can be removed from the gas stream introduced into the unit 5 depends on the amount of heavy hydrocarbons removed by the heavy hydrocarbon removal unit 3. The total amount of CO2 contained in the total gas stream admitted to the CO2 removal unit 5 is the sum of the CO2 contained in the gas from the gas source 2 after passing through the heavy hydrocarbon removal unit 3 and the CO2 contained in the purified biogas leaving the outlet 20 of the biogas purification unit 9.

The gas purged of heavy hydrocarbons leaves the heavy hydrocarbon removal unit 3 via the first outlet 6 and is provided to the CO2 removal unit 5 via the first inlet 10.

Advantageously, the pressure of the gas coming from the gas source 2 remains constant, for example at 45 barg. This pressure is kept high or constant during all steps of the purification process (a step of removing heavy hydrocarbons from said gas by the heavy hydrocarbon removal unit 3, a step of providing the gas purged of heavy hydrocarbons to a CO2 removal unit 5, and a CO2 removal step).

In parallel, the biogas purification unit 9 is supplied with raw biogas 18 via the inlet 16, wherein the biogas is purified to produce a purified biogas with a higher CH4 concentration and a lower CO2 concentration than the raw biogas admitted to the inlet 16. The purified biogas leaving the first outlet 20 and provided to the CO2 removal unit 5 via the first inlet 10.

The gas purged of heavy hydrocarbons and the purified biogas can be supplied to the CO2 removal unit 5 via the first inlet 10 independently, i.e. the two gas streams are combined only inside the unit. Alternatively, and as illustrated, the gas purged of heavy hydrocarbons and the purified biogas can be combined upstream of the CO2 removal unit 5, for example by way of a conduit for the gas purged of heavy hydrocarbons and a conduit for the purified biogas, and supplied to the unit 5 with a combined gas stream.

Advantageously, the purified biogas is compressed to the pressure of the gas coming from the gas source 2, for example to 45 barg, before being provided to the CO2 removal unit 5 or before being combined with the gas purged of heavy hydrocarbons upstream of the CO2 removal unit 5.

The plant 1 is configured to modulate the level of purification of the biogas according to the thermal energy produced by the boiler 11 and provided to the CO2 removal unit 5.

To produce purified methane having a predetermined CO2 concentration and exiting through the outlet 12 of the CO2 removal unit 5, the total CO2 content in the gas stream admitted to the unit 5 is adjusted according to the thermal energy available from the burning of heavy hydrocarbons within the boiler 11. In particular, the total CO2 content/quantity/concentration admitted to the CO2 removal unit 5 is adjusted by varying the degree of purification of the raw biogas in the biogas purification unit 9. The degree of biogas purification determines the CO2 concentration level in the purified biogas leaving the outlet 20 of the biogas purification unit 9.

The operation of the biogas purification unit 9 is optimized according to the energy available from the burning of the heavy hydrocarbons extracted at the heavy hydrocarbon removal unit 3. The plant 1 is configured so that the energy required for the operation of the CO2 removal unit 5 in the gas stream to reach a defined threshold is equal to the energy obtained by the burning of heavy hydrocarbons.

The CO2 content at the outlet from the biogas purification unit 9, i.e. the molar concentration of CO2 in biomethane, is determined by a function related to a certain number of parameters. A unique energy efficiency optimum is found as a function of the molar flow rates of the gas comprising natural gas and raw biogas as well as the concentrations of heavy hydrocarbons in the gas comprising natural gas and CO2 in raw biogas. Thus, the plant 1 according to the invention makes it possible to vary/modulate the degree of purification of the biogas in order to optimize the energy efficiency and the equipment costs (“CAPEX”) associated with the biogas purification unit 9.

For example, the biogas purification unit 9 is a membrane permeation treatment unit comprising one or more membrane separation units. Each membrane separation unit (also called a “stage”) may comprise one or more membranes connected in parallel. Several membrane stages (typically between 2 and 4) are required to achieve the purification rate of about 2% CO2 at the outlet of the unit 9. A less advanced purification allows this number of stages to be reduced, for example from 1 to 2.

In one embodiment example of the invention, the biogas purification unit 9 can operate in a fixed mode in which the biogas purification level is predetermined/fixed. This is applicable, for example, when the flow rate and the composition of the gas stream coming from the source 2, in particular the amount of heavy hydrocarbons provided to the boiler 11, is known beforehand.

In another embodiment example of the invention, the plant can be configured to modulate the level of biogas purification in a dynamic mode as a function of the amount of heavy hydrocarbons removed by the heavy hydrocarbon removal unit 3 or the thermal energy produced by the boiler 11 provided to the CO2 removal unit 5.

The plant 1 may comprise a device for continuously measuring or analysing the amount of heavy hydrocarbons provided to the boiler 11 or the thermal energy produced by the boiler 11 and provided to the CO2 removal unit 5. The plant 1 may comprise an element for sending a signal to the biogas purification unit 9 on the basis of the value measured/analysed by the measuring or analysing device. The biogas purification unit 9 can be configured to receive said signal to adjust the purification level so that the amount of CO2 remaining in the biomethane (treated biogas) at the outlet of the purification unit 9 is regulated so that the thermal power necessary for the removal of CO2 at a defined threshold of the gas admitted to the CO2 removal unit 5 corresponds to the thermal power generated by the burning of heavy hydrocarbons in the boiler 11.

Preferably, the CO2 removal unit 5 comprises or is an amine scrubbing unit. In the case where the CO2 removal unit 5 is an amine scrubbing unit, the thermal power required for CO2 removal corresponds to the thermal power required for amine regeneration. The CO2 removal unit 5 is thus configured to have an amine scrubbing capacity as a function of the thermal energy provided by the boiler 11.

In one embodiment example of the invention, the boiler 11 is configured to provide the heavy hydrocarbon removal unit 3 with thermal energy 25, for example by means of a heat transfer fluid. The thermal energy produced by the burning of heavy hydrocarbons in the boiler 11 can be provided in part 25 to the heavy hydrocarbon removal unit 3 and in part 23 to the CO2 removal unit 5. Excess energy after providing the energy necessary for CO2 removal 5 may be provided for at least partial removal 3 of heavy hydrocarbons.

As can be seen in FIG. 1, for this purpose, the CO2 removal unit 5 may comprise a third gas outlet 14 comprising a fuel and the boiler 11 may comprise a second fuel gas inlet 24 connected to the third outlet 14 of the CO2 removal unit 5. The CO2 removal unit 5 (especially when it is an amine scrubbing unit) can be purged (“flashed”) to remove CO2 with a stream comprising residual hydrocarbons. This gas comprising a fuel which leaves the third outlet 14 of the CO2 removal unit 5 can be provided to the boiler 11 via the second inlet 24.

The plant 1 may comprise a conduit, for example a bypass, intended to connect the first purified biogas outlet 20 as well to a natural gas network. For example, this may be a local, regional or national natural gas distribution network. The purified biogas leaving the first outlet 20 of the biogas purification unit 9 may have a purity corresponding to the biomethane specification sufficient to be reinjected into the natural gas network.

The biogas purification unit 9 may comprise a second CO2-rich gas outlet 28.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing i.e. anything else may be additionally included and remain within the scope of “comprising.” “Comprising” is defined herein as necessarily encompassing the more limited transitional terms “consisting essentially of” and “consisting of”; “comprising” may therefore be replaced by “consisting essentially of” or “consisting of” and remain within the expressly defined scope of “comprising”.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1. A plant for production of purified methane from natural gas and biogas, comprising, arranged in series in a gas circuit, a heavy hydrocarbon removal unit and a CO2 removal unit, the heavy hydrocarbon removal unit comprising:
a first inlet configured to be connected to a gas source comprising natural gas,
a first outlet for gas purged of heavy hydrocarbons, and
a second outlet for the removed heavy hydrocarbons,
the CO2 removal unit comprising:
a first inlet connected to the first outlet of the heavy hydrocarbon removal unit,
a first outlet for gas with reduced CO2 content, and
a second outlet for CO2-enriched effluent gases,
the plant further comprising:
a biogas purification unit comprising an inlet configured to be connected to a biogas source and a first purified biogas outlet connected to the first inlet of the CO2 removal unit, the biogas purification unit being configured to produce biogas or biomethane with a variable/defined CO2 concentration, and
a boiler for producing heat by combustion of heavy hydrocarbons, comprising a first inlet connected to the second outlet of the heavy hydrocarbon removal unit, the boiler being configured to produce a thermal power determined as a function of the amount of hydrocarbons removed and supplied by the heavy hydrocarbon removal unit, the boiler being fluidically connected to the CO2 removal unit to provide said unit with thermal energy produced for CO2 removal.

2. The plant according to claim 1, wherein the CO2 removal unit comprises or is an amine scrubbing unit.

3. The plant according to claim 1, wherein the CO2 removal unit is configured to have a capacity for removing a defined amount of CO2 from a gas stream, y, said capacity being a function of the thermal energy provided by the boiler.

4. The plant according to claim 1, wherein the plant is configured to measure or determine the thermal energy produced by the boiler and provided to the CO2 removal unit.

5. The plant according to claim 1, wherein the plant is configured to modulate the level of purification of the biogas as a function of the thermal energy produced by the boiler and provided to the CO2 removal unit.

6. The plant according to claim 1, wherein the boiler is configured to provide thermal energy to the heavy hydrocarbon removal unit.

7. The plant according to claim 1, wherein the CO2 removal unit comprises a third outlet for gas comprising a fuel and in that the boiler comprises a second inlet for fuel gas, connected to the third outlet of the CO2 removal unit.

8. The plant according to claim 1, wherein the biogas purification unit is a membrane separation unit.

9. The plant according to claim 1, wherein the plant comprises a conduit for connecting the first purified biogas outlet to a natural gas network.

10. The plant according to claim 1, wherein the biogas purification unit comprises a second CO2-rich gas outlet.

11. A process for production of purified methane from natural gas and biogas, the process comprising:
supplying gas comprising natural gas to a heavy hydrocarbon removal unit,
removing heavy hydrocarbons from said gas by the heavy hydrocarbon removal unit thereby producing a gas purged of heavy hydrocarbons,
providing the gas purged of heavy hydrocarbons to a CO2 removal unit, the CO2 removal unit ensuring CO2 removal by a process comprising heating,
supplying a biogas purification unit with biogas,
purifying biogas in the biogas purification unit to produce a purified biogas,
providing the purified biogas to the CO2 removal unit,
burning, in a boiler, the heavy hydrocarbons removed by the heavy hydrocarbon removal unit,
providing the thermal energy produced by the burning of the heavy hydrocarbons to the CO2 removal unit,
removing CO2 within the gas stream comprising the sum of the gas purged of heavy hydrocarbons and the purified biogas, the CO2 removal step being configured to reduce the CO2 concentration in the gas stream to a defined threshold,
in which the biogas purification step is configured to produce a purified biogas having a defined CO2 concentration, said concentration being chosen as a function of the total amount of CO2 to be removed from the gas stream in the CO2 removal unit in order to reach the CO2 concentration threshold.

12. The process according to claim 11, wherein the total amount of CO2 to be removed from the gas stream in the CO2 removal unit to reach the CO2 concentration threshold corresponds to the CO2 removal capacity of the removal unit, said capacity being determined by the thermal energy produced and provided by the boiler.

13. The process according to claim 11, wherein it comprises a step of determining the thermal energy produced by the boiler and available to the CO2 removal unit.

14. The process according to claim 11, wherein the purified biogas is compressed to the pressure of the gas purged of heavy hydrocarbons and combined with said gas to form the gas stream provided to the CO2 removal unit.
